SEQUENCE LISTING (9 sequences)

SEQ ID NO: 1 - protein, 226 aa, Homo sapiens
DERDRVQKKTFTKWVNKHLIKAQRHISDLYEDLRDGHNLISLLEVLSGDSLPREKGRMRFHKLQNVQIALDYLRHRQVKLVNIRNDDIADGNPKLTLGLIWTIILHFQISDIQVSGQSEDMTAKEKLLLWSQRMVEGYQGLRCDNFTSSWRDGRLFNAIIHRHKPLLIDMNKVYRQTNLENLDQAFSVAERDLGVTRLLDPEDVDVPQPDEKSIITYVSSLYDAMP

SEQ ID NO: 2 - protein, 227 aa, Homo sapiens
DERDRVQKKTFTKWVNKHLIKAQRHISDLYEDLRDGHNLISLLEVLSGDSLPREKGRMRFHKLQNVQIALDYLRHRQVKLVNIRNDDIADGNPKLTLGLIWTIILHFQISDIQVSGQSEDMTAKEKLLLWSQRMVEGYQGLRCDNFTSSWRDGRLFNAIIHRHKPLLIDMNKVYRQTNLENLDQAFSVAERDLGVTRLLDPEDVDVPQPDEKSIITYVSSLYDAMPR

SEQ ID NO: 3 - RNA, 21 nt, Artificial Sequence
  siRNA
ggaaugaugacaucgcugauu

SEQ ID NO: 4 - RNA, 21 nt, Artificial Sequence
  siRNA
ucagcgaugucaucauuccug

SEQ ID NO: 5 - DNA, 452 bp, Homo sapiens
ttcaccaagtgggtcaacaagcacctcatcaaggcccagaggcacatcagtgacctgtatgaagacctccgcgatggccacaacctcatctccctgctggaggtcctctcgggggacagcctgccccgggagaaggggaggatgcgtttccacaagctgcagaatgtccagattgccctggactacctccggcaccgccaggtgaagctggtgaacatcaggaatgatgacatcgctgacggcaaccccaagctgacccttggcctcatctggacaatcattctgcacttccagatctcagatatccaggtgagtgggcagtcggaggacatgacggccaaggagaagctgctgctgtggtcgcagcgaatggtggaggggtaccagggcctgcgatgcgacaacttcacctccagctggagagacggccgcctcttcaatgccatcatcca

SEQ ID NO: 6 - protein, 8 aa, Artificial Sequence
  peptide
AVIYGGVQ

SEQ ID NO: 7 - protein, 6 aa, Artificial Sequence
  peptide
WPYKKY

SEQ ID NO: 8 - protein, 11 aa, Artificial Sequence
  peptide
AVIYGGVQGGC

SEQ ID NO: 9 - protein, 9 aa, Artificial Sequence
  peptide
WPYKKYGGC

(19) United States
(12) Patent Application Publication
(10) Pub. No.: US 2023/0303672 A1
(43) Pub. Date: Sep. 28, 2023

(54) METASTATIC CANCER TREATMENT OR PREVENTION

(71) Applicant: OXFORD BROOKES UNIVERSITY, Oxford, Oxfordshire (GB)

(72) Inventors: Susan BROOKS, Oxford, Oxfordshire (GB); David CARTER, Oxford, Oxfordshire (GB); Ellie BEAMAN, Oxford, Oxfordshire (GB)

(73) Assignee: OXFORD BROOKES UNIVERSITY, Oxford, Oxfordshire (GB)

(21) Appl. No.: 18/020,749

(22) PCT Filed: Aug. 13, 2021
(86) PCT No.: PCT/GB2021/052109

§ 371 Date: Feb. 10, 2023

(30) Foreign Application Priority Data
Aug. 14, 2020 (GB) 2012761.9

(51) Int. Cl. C07K 16/18; A61K 39/395; A61P 35/04

(52) U.S. Cl. CPC C07K 16/18; A61K 39/3955; A61P 35/04; A61K 2039/505

(57) ABSTRACT

The invention relates to agents which prevent or reduce the interaction between actin and one or more of its interacting partners, for use in treating or preventing metastatic cancer in a subject in need thereof. The invention also relates to a method of treating or preventing metastatic cancer, or a method of preventing or reducing epithelial cell to endothelial cell adhesion, comprising administering an agent which prevents or reduces the interaction between actin and one or more of its interacting partners.

### TECHNICAL FIELD

The present invention relates to the treatment or prevention of metastatic cancer.

### INTRODUCTION

Epithelial cell cancers, known as carcinomas, account for 80 to 90 percent of all cancer cases. One such carcinoma, breast cancer, is the second most common cancer type in the world and accounts for over 10% of all cancers. Breast cancer is the most common cancer in women, with more than two million women being diagnosed annually, and over 500,000 patients dying annually of the disease.

Metastatic dissemination of a primary tumour is the leading cause of cancer related mortality, accounting for up to 90% of solid tumour cancer deaths. Metastasis occurs when cancer cells acquire a migratory epithelial-to-mesenchymal transition (EMT) phenotype, initiated from groupings of cells that disseminate from primary tumours. An invasive phenotype of such cells is a fundamental property, which correlates with their invasion to the endothelial vascular layer in the early stages of metastasis. Additionally, patients that are diagnosed with tumours that have metastasised have a 5-year survival rate of only 22%.

Therefore there is a need to develop new and improved prophylactic and therapeutic treatments for metastatic cancer. The present invention fulfils these needs and further provides other related advantages.

### SUMMARY OF INVENTION

In a first aspect, the invention provides an agent which prevents or reduces the interaction between actin and one or more of its interacting partners, for use in treating or preventing metastatic cancer in a subject in need thereof.

In a second aspect, the invention provides a method of treating or preventing metastatic cancer, comprising administering to a subject in need thereof: a therapeutically effective amount of one or more agents which prevent or reduce the interaction between actin and one or more of its interacting partners. The agent may be formulated in a pharmaceutical composition disclosed herein.

In a third aspect, the invention provides a pharmaceutical composition comprising an agent of the invention for use in treating or preventing metastatic cancer in a subject in need thereof.

In a fourth aspect, the invention provides a method of preventing or reducing epithelial cell to endothelial cell adhesion, comprising administering to a subject, or to one or more cells, one or more agents which prevent or reduce the interaction between actin and one or more of its interacting partners.

In a fifth aspect, there is provided an anti-actin antibody for use in treating or preventing metastatic cancer in a subject in need thereof.

The invention is in part based on the finding of a positive correlation between the presence and/or level of actin on the surface of epithelial cancer cells and/or extracellular vesicles derived therefrom, and the metastatic potential of these cancer cells. The invention is also based on the finding that disrupting the interaction between actin on the surface of epithelial cancer cells and/or extracellular vesicles derived therefrom, with its interacting partners such as plectin or an actin binding fragment of plectin, reduces epithelial cell to endothelial cell adhesion. Reducing or preventing such interactions, for example by blocking the interaction or reducing the expression of one or more of the proteins which is directly or indirectly involved in the interaction, provides a means of preventing or treating metastasis. Cells which have undergone epithelial-mesenchymal transition (EMT) are then unable to migrate via blood vessels to invade secondary sites where metastatic tumours progress. Such a preventative or therapeutic treatment is advantageous in the prevention of a secondary metastatic tumour being established, or in extending the life expectancy of a patient who already has a metastatic cancer. The invention also allows the targeting of metastatic cells with elevated expression of actin on their surface with one or more therapeutics.

In any aspect, the actin may be one or more of beta-actin, alpha actin or gamma actin. The actin may be all of beta-actin, alpha actin and gamma actin. Preferably, the actin is beta actin. Actin may refer to beta-actin which is the protein encoded by the ACTB gene (Uniprot P60709). Actin may refer to alpha-actin which is the protein encoded by the ACTA1 (Uniprot P68133) or ACTA2 (Uniprot P62736) genes. Actin may refer to gamma-actin which is the protein encoded by the ACTG1 (Uniprot P63261) or ACTG2 genes (Uniprot P63267).

Accession numbers, where given, relate to those identifiable using UniProt (Universal Protein Resource), a comprehensive catalogue of information on proteins (‘UniProt: a hub for protein information’ Nucleic Acids Res. 43: D204-D212 (2015)).

In any aspect, the one or more interacting partners may comprise or consist of plectin (Uniprot Q15149) or an actin binding fragment thereof. The actin binding fragment thereof may comprise or consist of the actin binding domain (ABD) of plectin (SEQ ID NO 1 or SEQ ID NO: 2).

The sequence of SEQ ID NO: 1 is:
DERDRVQKKTFTKWVNKHLIKAQRHISDLYEDLRDGHNLISLLEVLSGDS
LPREKGRMRFHKLQNVQIALDYLRHRQVKLVNIRNDDIADGNPKLTLGLI
WTIILHFQISDIQVSGQSEDMTAKEKLLLWSQRMVEGYQGLRCDNFTSSW
RDGRLFNAIIHRHKPLLIDMNKVYRQTNLENLDQAFSVAERDLGVTRLLD
PEDVDVPQPDEKSIITYVSSLYDAMP.
The sequence of SEQ ID NO: 2 is:
DERDRVQKKTFTKWVNKHLIKAQRHISDLYEDLRDGHNLISLLEVLSGDS
LPREKGRMRFHKLQNVQIALDYLRHRQVKLVNIRNDDIADGNPKLTLGLI
WTIILHFQISDIQVSGQSEDMTAKEKLLLWSQRMVEGYQGLRCDNFTSSW
RDGRLFNAIIHRHKPLLIDMNKVYRQTNLENLDQAFSVAERDLGVTRLLD
PEDVDVPQPDEKSIITYVSSLYDAMPR.

In any aspect, the interacting partner may be present on the surface of a cell, such as an endothelial cell. The interacting partner may be present on the surface of an extracellular vesicle (EV).

The interaction between actin and plectin, or between an actin binding fragment of plectin, may be direct, in that actin directly interacts with plectin or an actin binding fragment thereof, at the surface of a cell, or at the interface between two cells such as endothelial and epithelial cell. Where the interaction is at the interface between two cells, preferably the actin is on the surface of an epithelial cell, and the plectin or an actin binding fragment thereof is on the surface of an endothelial cell.

The interaction between actin and plectin or an actin binding fragment thereof may be indirect, in that actin indirectly interacts with plectin or an actin binding fragment thereof via intermediate proteins, which may be present at the surface of a cell.

The agent may bind to actin. The agent may bind to an actin interacting partner, such as plectin or an actin binding fragment thereof. The agent may bind to actin and to an actin interacting partner, such as plectin or an actin binding fragment thereof (e.g. the agent may be bi-specific).

In any aspect, the agent may be a polypeptide, an antigen binding polypeptide, a nucleic acid molecule, or a small molecule.

Agents

An agent which is a polypeptide may comprise or consist of actin, as defined above, or a sequence with about 90% or more, about 95% or more, about 96% or more, about 97% or more, about 98% or more, about 99% or more identity to actin as defined above.

The polypeptide may comprise or consist of plectin or an actin binding fragment thereof as defined above, or a sequence with about 90% or more, about 95% or more, about 96% or more, about 97% or more, about 98% or more, about 99% or more identity to plectin or an actin binding fragment thereof as defined above.

The polypeptide may comprise or consist of the ABD of plectin (SEQ ID NO: 1 or SEQ ID NO: 2) or a sequence with about 90% or more, about 95% or more, about 96% or more, about 97% or more, about 98% or more, about 99% or more identity to SEQ ID NO: 1 or SEQ ID NO: 2.

The agent may be a polypeptide which binds to actin. The agent may be a polypeptide which binds to an actin interacting partner, such as plectin or an actin binding fragment thereof. The agent may bind to actin and to an actin interacting partner, such as plectin or an actin binding fragment thereof (e.g. the agent may be bi-specific).

The polypeptide may comprise or consist of the sequence of AVIYGGVQ (SEQ ID NO: 6).

The polypeptide may comprise or consist of the sequence of WPYKKY (SEQ ID NO: 7).

The polypeptide may further comprise or consist of the sequence GGC and be conjugated with HSA. Thus, polypeptide may comprise or consist of the sequence of AVIYGGVQGGC-HAS (SEQ ID NO: 8) or WPYKKYGGC-HAS (SEQ ID NO: 9). HSA as used herein refers to human serum albumin.

The polypeptide may be recombinant.

An agent which is an antigen binding polypeptide may refer to an antibody or antigen binding fragment thereof, or a T-cell Receptor (TCR). An antigen binding polypeptide has specificity for a target antigen.

An antibody or antigen binding fragment thereof may refer to monoclonal antibodies, multispecific antibodies, human antibodies, humanized antibodies, synthetic antibodies, chimeric antibodies, polyclonal antibodies, camelized antibodies, single-chain Fvs (scFv), single-chain antibodies, immunologically active antibody fragments (e.g., antibody fragments capable of binding to an epitope, e.g., Fab fragments, Fab′ fragments, F(ab′)2 fragments, Fv fragments, fragments containing a VL and/or VH Domain, or that contain 1, 2, or 3 of the complementary determining regions (CDRs) of such VL Domain (i.e., CDRL1, CDRL2, and/or CDRL3) or VH Domain (i.e., CDRH1, CDRH2, and/or CDRH3)) that specifically bind an antigen, etc., bi-functional or multi-functional antibodies, disulfide-linked bispecific Fvs (sdFv), intrabodies, and diabodies, and epitope binding fragments of any of the above. In particular, the term “antibody” is intended to encompass immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

An antibody or antigen binding fragment thereof may include a bispecific antibody, comprising two different variable regions, each of which specifically bind different epitopes, either on the same or on different antigens.

The target antigen may be an antigen on a protein which contributes to the interaction between plectin and actin. The protein may be actin, such as beta actin. The protein may be plectin, or an actin binding fragment thereof, comprising or consisting of the ABD of plectin.

The antigen binding polypeptide may be an anti-actin antibody. The antigen binding polypeptide may be an anti-beta-actin antibody. The antigen binding polypeptide may bind to all alpha, beta and gamma isoforms of actin. The antigen binding polypeptide may be an anti-plectin antibody. The antigen binding polypeptide may be an antibody which binds to the ABD of plectin.

A bispecific antibody may target two different antigens, either on the same protein or different proteins. For example, the bispecific antibody may bind to (a) actin or plectin (or an actin binding fragment thereof, and (b) an immune cell marker such as a dendritic cell, macrophage, or NK cell surface marker. This would allow the bispecific antibody to bring certain immune cells, important in the host's natural immune response to tumour cells, into close proximity with cells which have actin and/or plectin or an actin binding fragment thereof on their surface.

A range of such immune cell surface markers are known in the art for the skilled person to choose from. Non limiting examples of dendritic cell markers include CD11b, CD11c, CD103, CD14, CD8-alpha, or an MHC Class II molecule. Non limiting examples of macrophage markers include CD14, CD16, CD64, CD68, CD71 and CCRS. Non limiting examples of NK Cell markers include CD56, CD94, the KIR family receptor, NKG2A, NKG2D, NKp30, NKp44, NKp46, NKp80.

An anti-actin antibody may induce antibody-dependent cellular cytotoxicity of metastatic cells which have increased actin on their cell surface. An anti-actin antibody may be conjugated to a known agent which induces cell death, such as immunomodulators, radioactive compounds, enzymes, chemotherapeutic agents or cytotoxic agents.

Antibodies or antigen binding fragments thereof, including polyclonal, monoclonal and bispecific antibodies, can be prepared using conventional immunization techniques, as will be generally known to those of skill in the art.

The antibody may be anti-plectin antibody clone PN753 (Cosmo Bio).

The antibody may be anti-plectin antibody clone PN643 (Kerafast).

The antibody may be anti-plectin antibody clone Q0754 (Creative Diagnostics).

The antibody may be anti-actin antibody clone mAbcam 8226 (Abcam).

The antibody may be anti-actin antibody clone 15G5A11/E2 (ThermoFisher).

The antibody may be anti-actin antibody clone 2-2.1.14.17 (Sigma-Aldrich).

The antibody may be anti-actin antibody clone 2A3 (BioRad).

An agent which is a nucleic acid molecule may be a DNA or RNA molecule.

The nucleic acid molecule may bind to actin or one or more of its interacting partners, or DNA or RNA which encodes actin or one or more of its interacting partners.

The nucleic acid molecule may be a DNA or RNA aptamer which binds actin, such as beta actin or all isoforms of actin. The nucleic acid molecule may be a DNA or RNA aptamer which binds plectin or an actin binding fragment thereof, such as the ABD of plectin.

The nucleic acid molecule may prevent or reduce the expression of actin or one or more of its interacting partners, such as an oligonucleotide, an siRNA or an shRNA. The oligonucleotide, siRNA or shRNA may prevent or reduce the expression of actin such as beta actin or all isoforms of actin. The siRNA or shRNA may prevent or reduce the expression of plectin or an actin binding fragment thereof, such as the ABD of plectin. In an embodiment, the nucleic acid molecule is an siRNA which reduces the expression of plectin.

In an embodiment, there is provided a mixture of siRNAs which reduce the expression of plectin. The mixture may comprise at least one or more, such as one, two, three four, five, six, seven, eight, nine or ten or more siRNAs which reduce the expression of plectin. The at least one or more siRNA may comprise or consist of the sequence of GGAAUGAUGACAUCGCUGAUU (SEQ ID NO: 3) and/or the sequence of UCAGCGAUGUCAUCAUUCCUG (SEQ ID NO: 4).

In an embodiment, the at least one or more siRNAs may target at least a part of the sequence of:

(SEQ ID NO: 5)
TTCACCAAGTGGGTCAACAAGCACCTCATCAAGGCCCAGAGGCACATCAG
TGACCTGTATGAAGACCTCCGCGATGGCCACAACCTCATCTCCCTGCTGG
AGGTCCTCTCGGGGGACAGCCTGCCCCGGGAGAAGGGGAGGATGCGTTTC
CACAAGCTGCAGAATGTCCAGATTGCCCTGGACTACCTCCGGCACCGCCA
GGTGAAGCTGGTGAACATCAGGAATGATGACATCGCTGACGGCAACCCCA
AGCTGACCCTTGGCCTCATCTGGACAATCATTCTGCACTTCCAGATCTCA
GATATCCAGGTGAGTGGGCAGTCGGAGGACATGACGGCCAAGGAGAAGCT
GCTGCTGTGGTCGCAGCGAATGGTGGAGGGGTACCAGGGCCTGCGATGCG
ACAACTTCACCTCCAGCTGGAGAGACGGCCGCCTCTTCAATGCCATCATC
CA,

which is a cDNA target sequence of plectin.

The nucleic acid molecule may be encoded in a DNA vector, such as a plasmid.

The nucleic acid molecule may be packaged into a viral vector. The viral vector may be an adenoviral vector, or a lentiviral vector.

Nucleic acid molecules, DNA vectors and viral vectors as described herein can be designed and prepared using conventional genetic and molecular biology techniques.

An agent which is a small molecule may refer to a molecule which can bind to actin or one or more of its interacting partners. The small molecule may bind to actin, such as beta actin or all isoforms of actin. The small molecule may bind to plectin, or an actin binding fragment thereof, such as the ABD of plectin. The small molecule may comprise a sugar which binds to plectin.

A pharmaceutical composition may comprise or consist of one or more different agents disclosed herein, such as one, two or more, three or more, four or more, five or more, six or more, or seven or more of the agents disclosed herein and a pharmaceutically acceptable excipient or carrier.

The pharmaceutical composition may comprise an agent disclosed herein, such as an anti-actin antibody, plectin or an actin binding fragment thereof such as the ABD of plectin.

The pharmaceutical composition may comprise an anti-actin antibody. The pharmaceutical composition may comprise plectin or a polypeptide with a sequence with about 90% or more, about 95% or more, about 96% or more, about 97% or more, about 98% or more, about 99% or more identity to plectin. The pharmaceutical composition may comprise the ABD of plectin (SEQ ID NO: 1 or SEQ ID NO: 2) or a polypeptide with about 90% or more, about 95% or more, about 96% or more, about 97% or more, about 98% or more, about 99% or more identity to SEQ ID NO: 1 or SEQ ID NO: 2.

The pharmaceutical composition may comprise an shRNA targeting actin, such as beta actin. The pharmaceutical composition may comprise an shRNA targeting plectin or an actin binding fragment thereof. The pharmaceutical composition may comprise an shRNA targeting the ABD of plectin.

The pharmaceutical composition may comprise a viral vector encoding an agent of the invention. The pharmaceutical composition may comprise a lentiviral vector comprising or encoding an shRNA of the invention.

The pharmaceutical composition may comprise two different agents disclosed herein. A pharmaceutical composition comprising two different agents disclosed herein may comprise or consist of a first agent which binds to actin, and a second agent which binds to plectin or an actin-binding fragment thereof, such as the ABD of plectin.

The pharmaceutical composition may comprise an anti-actin antibody and a polypeptide encoding plectin or a sequence with about 90% or more, about 95% or more, about 96% or more, about 97% or more, about 98% or more, about 99% or more identity to plectin. The pharmaceutical composition may comprise an anti-actin antibody and a polypeptide of SEQ ID NO: 1 or SEQ ID NO: 2, or a sequence with about 90% or more, about 95% or more, about 96% or more, about 97% or more, about 98% or more, about 99% or more identity to SEQ ID NO: 1 or SEQ ID NO: 2.

In any aspect, the agent or pharmaceutical composition may be administered to the subject prior to diagnosis of metastatic cancer, or after diagnosis of metastatic cancer. The agent or pharmaceutical may be administered to the subject indefinitely or for a specified period of time. The agent or pharmaceutical may be administered at regular intervals.

The pharmaceutical composition may further comprise one or more carriers or excipients.

For example, an excipient can give form or consistency, or act as a diluent. Suitable excipients include, but are not limited to, stabilizing agents, wetting and emulsifying agents, salts for varying osmolarity, encapsulating agents, buffers, and skin penetration enhancers. The compositions can be in any suitable form, for example tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. Such compositions may be prepared by any known method, for example by admixing the active ingredient with the carrier(s) or excipient(s) under sterile conditions.

Suitable formulations for parenteral administration include aqueous solutions of the active compounds in water-soluble form, for example, water-soluble salts. In addition, suspensions of the active compounds as appropriate for oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances that increase the viscosity of the suspension and include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulation may be used simultaneously to achieve systemic administration.

Suitable formulations for oral administration include hard or soft gelatin capsules, pills, tablets, including coated tablets, elixirs, suspensions, syrups or inhalations and controlled release forms thereof. Generally, these agents are formulated for administration by injection (e.g., intraperitoneally, intravenously, subcutaneously, intramuscularly, etc.), although other forms of administration (e.g., oral, mucosal, etc.) can be also used. Accordingly, agents of the invention are preferably combined with pharmaceutically acceptable vehicles such as saline, Ringer's solution, dextrose solution, and the like.

The pharmaceutical compositions can also be formulated so as to provide quick, sustained or delayed release of their active ingredients after administration to the patient by employing procedures known in the art. The physical and chemical characteristics of the compositions of the invention may be modified or optimized according to the skill in the art, depending on the mode of administration and the particular disease or disorder to be treated. The compositions may be provided in unit dosage form, a sealed container, or as part of a kit, which may include instructions for use and/or a plurality of unit dosage forms.

Where more than one agent is to be administered, the agents may be formulated together in the same formulation or may be formulated into separate pharmaceutical compositions. The separate compositions may be administered concurrently, sequentially or separately.

A variety of administration routes for the agents or pharmaceutical compositions of the invention are available. The particular mode selected will depend upon the particular agent or composition selected, whether the administration is for prevention, or treatment of disease, the severity of the medical disorder being treated and dosage required for therapeutic efficacy. The methods of this invention may be practiced using any mode of administration that is medically acceptable, and produces effective levels of the active compounds without causing clinically unacceptable adverse effects. Such modes of administration include, but are not limited to, oral, buccal, sublingual, inhalation, mucosal, rectal, intranasal, topical, ocular, periocular, intraocular, transdermal, subcutaneous, intra-arterial, intravenous, intramuscular, parenteral, or infusion methodologies. In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

As used herein, the term “therapeutically effective amount” refers to the total amount of the agent or each active component of the pharmaceutical composition or method that is sufficient to provide patient benefit, i.e., prevention or amelioration of the condition to be treated, a reduction in symptoms, an increase in rate of healing, or a detectable change in the levels of a substance in the treated or surrounding tissue. When applied to an individual active ingredient, administered alone, the term refers to that ingredient alone. When applied to a combination, the term refers to combined amounts of the active ingredients that result in the therapeutic effect, whether administered in concurrently, sequentially or separately.

The precise dose to be employed in the formulations of the present invention may depend on the route of administration, and the seriousness of the condition, and should be decided according to the judgment of the practitioner and each patient's circumstances and can be determined by standard clinical techniques. Effective doses may be extrapolated from dose-response curves derived from in vitro or animal model test systems. The particular dosage regimen, i.e., dose, timing and repetition, will thus depend on the particular individual and that individual's medical history, as well as the route of administration.

The agents or compositions may be delivered at intervals ranging from about 24 hrs to about 2 days, to about 1 week, to about 2 weeks, to about 3 weeks to about 1 month to about 2 months, to about 3 months, to about 4 months, to about 5 months, to about 6 months. The scheduling of such dosage regimens can be optimized by the clinician.

The agents or compositions may be administered using a treatment regimen comprising one or more doses, wherein the treatment regimen is administered over 2 days, 3 days, 4 days, 5 days, 6 days or 7 days, 14 days, 30 days.

Combination

The agent, pharmaceutical composition or method of treatment of the invention may be combined with other known therapies for the treatment or prevention of cancer including metastatic cancer, autoimmune disease, inflammation, or infectious disease, including, current standard and experimental chemotherapies, hormonal therapies, biological therapies, immunotherapies, radiation therapies, or surgery.

The skilled person will be able to identify a suitable known treatment for metastatic cancer and will understand that that the known treatment for metastatic cancer will vary depending on how extensive and advanced the metastatic disease is likely to be and the molecular profile of the cancer. For example, a suitable treatment may be adjuvant chemotherapy, wherein the specific treatment and/or regime depends on the characteristics of the specific tumour of the subject. As an example, known treatments for metastatic cancer may be treatments known for use in treating metastatic breast cancer, such as Herceptin or another Her2 blocking monoclonal antibody treatment.

As used herein, the term “combination” or “combined with” as it relates to therapy, refers to the use of more than one therapeutic. The use of the term does not restrict the order in which agents or pharmaceutical compositions are administered to a subject in need thereof.

Subject in Need Thereof

The subject in need thereof of any aspect of the invention may have been diagnosed with cancer. The cancer may be an epithelial cell cancer. The epithelial cell cancer may be a breast cancer, prostate cancer, colorectal cancer, thyroid cancer, lung cancer or a melanoma.

The subject in need thereof of any aspect of the invention may have been diagnosed with metastatic cancer. The metastatic cancer may be derived from any cancer, such as lung cancer; prostate cancer; renal cancer; multiple myeloma; thyroid cancer; cancers of the head and neck; cancers of the digestive tract including stomach cancer, colon cancer, colorectal cancer, and liver cancer; malignant tumours of the female reproductive organs including ovarian cancer, endometrial cancer, and cervical cancer; bladder cancer; brain tumours including neuroblastoma; sarcoma; osteosarcoma; and skin cancers such as melanoma. Preferably the cancer is prone to metastasis. The metastatic cancer may be derived from an epithelial cancer, such as breast cancer, prostate cancer, colorectal cancer, thyroid cancer, lung cancer or a melanoma.

Identification of Subjects Suitable for Treatment

Membrane-bound actin may be detected or measured in a sample obtained from the subject prior to, concurrently with, or after treatment.

The membrane-bound actin may be detected or measured in the membrane of a cell, such as an epithelial cancer cell. The membrane bound actin may be detected or measured in the membrane of an extracellular vesicle (EV). The membrane bound actin may be detected or measured in both the membrane of a cell and in the membrane of an extracellular vesicle.

In any aspect, an EV may include, but is not limited to, exosomes, exomeres, microvesicles, and apoptotic bodies.

If actin is detected or measured at a level equal to or above the level of actin detected or measured in a reference sample, the subject may be determined to be suitable for, or likely to benefit from treatment with an agent of pharmaceutical composition of the invention.

The reference sample or sample obtained from the subject refers to a sample of biological material obtained from a subject, e.g., a human subject, including tissue, a tissue sample, a biopsy, a cell sample, a tumour sample, a stool sample and a sample of biological fluid, e.g., plasma, serum, blood, urine, lymphatic fluid, ascites, saliva.

In an embodiment, the sample is a tissue sample, for example from a biopsy of breast tissue from a subject. In an embodiment, the sample is a blood sample obtained from a subject.

The membrane-bound actin may be detected or measured from the plasma membrane of a breast cancer epithelial cell from a solid breast tissue biopsy obtained from the subject.

The membrane-bound actin may be detected or measured from the membrane of an extracellular vesicle isolated from a blood sample obtained from the subject.

The reference sample may be a positive or negative reference sample.

When the reference sample is a positive reference sample, the determination may be made when the level of membrane-bound actin detected or measured in that sample is equal to or higher than the level of membrane-bound actin detected or measured in the reference sample.

When the reference sample is a negative reference sample, the determination may be made when the level of membrane-bound actin detected or measured in that sample is higher than the level of membrane-bound actin detected or measured in the reference sample.

The magnitude of difference may depend on the relationship between the sample obtained from the subject and the reference sample, and the characteristics of each sample. For example, the determination may be made when the level of membrane-bound actin detected or measured in the sample obtained from the subject is at least about 5% more than, at least about 10% more than, at least about 20% more than, at least about 50% more than, at least about 100% more than, at least about 200% more than, at least about 300% more than, at least about 400% more than, at least about 500% more than, at least about 1000% more than, at least about 2000% more than, at least about 5000% more than, about at least 10,000% more than, at least 20,000% or more, at least 50,000% more, at least 100,000% more, at least 500,000% more, at least 1,000,000% more than the level of membrane-bound actin detected or measured in the reference sample. Alternatively or additionally the determination may be made when the level of membrane-bound actin detected or measured in the sample obtained from the subject is at least about at least about 1-fold or more, at least about 2-fold or more, at least about 3-fold or more, at least about 4-fold or more, at least about 5-fold or more, at least about 10-fold or more, at least about 20-fold or more, at least about 50-fold or more, at least about 100-fold or more, at least about 250-fold or more, at least about 500-fold or more, at least about 1000-fold or more, at least about 5000-fold or more, at least about 10,000-fold or more, at least about 20,000-fold or more, at least about 50,000-fold or more, at least about 100,000-fold or more, at least about 500,000-fold or more higher than the level of membrane-bound actin detected or measured in the reference sample.

The sample obtained from the subject may comprise epithelial cells, and/or EVs, and/or circulating tumour cells, such as epithelial cells. If the sample comprises epithelial cells and/or EVs, the detection or measurement of membrane-bound actin may be carried out on these EVs, which may be first isolated, identified or separated from the remaining sample, if required.

Similarly circulating tumour cells may be isolated, identified or separated from the remaining sample, if required. If the cells and/or EVs are to be isolated, identified or separated from the remaining sample, one or more suitable biomarkers may be used. For example, to isolate, identify or separate epithelial cells from a sample, an epithelial anti-EpCAM antibody may be used. Similarly, to isolate or separate EVs from a sample, one or more of anti-CD9, anti-CD63 and/or anti-CD81 antibodies may be used. To isolate, identify or separate circulating tumour cells from a sample, a means to detect one or more of EpCAM, ER, PR, EGFR, HER2, TOP2A may be used (Nadal et al. Breast Cancer Research 2012, 14:R71). A means for detecting membrane-bound actin may be an actin binding polypeptide, such as an anti-actin antibody. A means as defined above may be a DNA or RNA molecule which is capable of binding the target, such as an aptamer. The skilled person will be able to choose any set of reagents which are available in the art to isolate cells and/or EVs from a sample. The exact reagents chosen may depend on the nature of the sample, for example specific reagents may be chosen which are particularly compatible with blood samples. The means for detecting or measuring membrane-bound actin and/or the means for identifying the cell type of interest may be provided bound to a solid support, such as a column matrix, an array, or well of a microtiter plate. Alternatively, the support can be provided as a separate element.

### General Definitions

The skilled person will understand that a number of methods or techniques in the art can be used to detect or measure the level of membrane-bound actin. Suitable techniques include immunohistochemistry, immunocytochemistry, flow cytometry, ELISA, lateral flow assay, western blot following biochemical or mechanical separation/isolation of membranes, dot-blots, slot-blots, mass spectrometry. The invention may therefore be widely adopted at many hospitals or laboratories globally, without the need for highly specialised equipment or training.

The skilled person will understand that a number of methods or techniques in the art can be used to isolate EVs from a sample (reviewed in Konoshenko et al., 2018, BioMed. Res. Intl.).

Actin as referred to herein is the protein which is endogenous to the host species. In humans, for example, actin may refer to beta-actin which is the protein encoded by the ACTB gene. Actin may refer to alpha-actin which is the protein encoded by the ACTA1 or ACTA2 genes. Actin may refer to gamma-actin which is the protein encoded by the ACTG1 or ACTG2 genes.

“Membrane-bound actin” as used herein refers to actin, such as beta-actin which is present or detectable on the surface of a cell, preferably in/on the plasma membrane of the cell, or which is present or detectable on the surface of an extracellular vesicle. The actin may have post-translational modifications such as glycosylation marks. The actin may be detectable without having to disrupt, chemically or mechanically, the cell and/or EV. The actin may be detectable in membranous fractions of cells and/or EVs that have been mechanically and/or chemically disrupted, wherein said fractions comprise membrane only of the plasma membrane of cells or of the EV. The actin which is detected or measured may be an integral membrane protein or a peripheral membrane protein which interacts with an integral membrane protein.

The term “patient” or “subject,” as used interchangeably herein, refers to any mammal, e.g., a human. Non-limiting examples of non-human mammals include non-human primates, dogs, cats, mice, rats, guinea pigs, rabbits, fowl, pigs, horses, cows, goats, sheep, etc.

A sample which is described as a “biopsy” can be obtained by conventional methods, using methods well known by the persons skilled in related medical techniques. The methods for obtaining a biopsy sample include splitting a tumour into large pieces, or microdissection, or other cell separating methods known in the art. The tumour cells can additionally be obtained by means of cytology through aspiration with a small gauge needle. To simplify sample preservation and handling, samples can be fixed in formalin and soaked in paraffin or first frozen and then soaked in a tissue freezing medium such as OCT compound by means of immersion in a highly cryogenic medium which allows rapid freezing. Other methods to preserve and/or process samples are known to those skilled in the art.

The sample can be manipulated to isolate cell types of interest or to isolate EVs. For example, a marker for a cell type of interest in a tissue sample or biopsy can be used to isolate or separate such cells. An example is the use of an EpCAM binding polypeptide to identify and/or separate epithelial cells from tissue taken in a biopsy, before membrane-bound actin, such as beta-actin, is detected or the level determined. Another example is the use of an EpCAM binding polypeptide to identify and/or separate EVs derived from epithelial cells from other EVs and/or biological material in the sample.

A reference sample may refer to an equivalent sample, for example the same tissue and/or cell type as the sample obtained from the subject or from other subjects, or from a cell line which closely resembles the sample type obtained from the subject.

A “negative reference sample” may refer to a reference sample which is non-cancerous, or which is cancerous/immortalised but which is known to have not developed or to not develop metastasis. For example, a negative reference sample may be a corresponding sample, such as a biopsy sample, from the area surrounding a tumour of the subject, or corresponding tissue from one or more healthy subjects. A negative reference sample may also be a cell line which is known to be non-metastatic in nature and which is of the same or similar nature to the sample obtained for the subject, for example a breast epithelial cell line which is known to be low in invasiveness such as BT474 cells (Lasfargues et al., 1978, J. Nat. Cancer Institute, 61(4): 967-978) as a negative reference sample for a breast tissue sample obtained from the subject, or HMT3522 Human breast epithelial cells isolated from benign fibrocystic breast tissue (Briand et al, 1987, In Vitro Cell and Dev. Biol., 23 (3): 181-188).

Where the presence or level of membrane-bound actin is to be detected or measured in EVs, a negative reference sample may refer to EVs isolated from one or more subjects who do not have cancer, or who have/had cancer and have not or did not develop metastasis, or from a comparative cell line which is known to be low in invasiveness, as above.

Thus, the level of membrane-bound actin detected or measured in such a negative reference sample acts as a negative control.

A “positive reference sample” may refer to a reference sample which is cancerous, which is known to be invasive, and/or which is known to have developed or to be likely to develop metastasis. For example, a positive reference sample may be a corresponding sample, such as a biopsy sample, from one or more subjects known to have developed metastasis. A positive reference sample may also be a cell line which is known to be invasive and/or metastatic in nature and which is of the same or similar nature to the sample obtained for the subject, for example a breast epithelial cell line which is known to be high in invasiveness such as MCF7 cells (Soule et al., 1973, J. Nat. Cancer Institute, 51(5): 1409-14-16) as a positive reference sample for a breast tissue sample obtained from the subject.

Where the presence or level of membrane-bound actin is to be detected or measured in EVs, a positive reference sample may refer to EVs isolated from one or more subjects who have/had cancer, and who developed metastasis, or from a comparative cell line which is known to be high in invasiveness, as above.

Thus, the level of membrane-bound actin detected or measured in such a positive reference sample acts as a positive control.

A sample from a subject who has been diagnosed with cancer and in which higher levels of membrane-bound actin are measured than in those of the above reference samples may be identified as being suitable to receive the agents, pharmaceutical compositions or methods of the invention.

The levels of actin on the surface of an EV can also be used to monitor therapeutic efficacy and tumour recurrence.

The skilled person could consult a database in which the level of membrane-bound actin in a given sample type has been recorded from subjects with confirmed metastasis or no recorded metastasis, or from cell lines which are known to be metastatic or non-metastatic in nature. The skilled person is easily able to identify such metastatic or non-metastatic cell lines, where appropriate.

In any aspect or embodiment, the cancer may be an epithelial cell cancer, such as breast cancer.

The method of any aspect of the invention may be in vivo, ex vivo or in vitro. In an embodiment, the method is an in vitro method.

In an aspect the invention provides a method of preventing or reducing epithelial cell to endothelial cell adhesion comprising administering to a subject, or to one or more cells, one or more agents of the invention. One or more agents may be administered to epithelial cells and/or to endothelial cells. The one or more agents which are administered to epithelial cells may comprise or consist of an anti-actin antibody, and/or the one or more agents which are administered to endothelial cells may comprise or consist of an anti-plectin antibody. Alternatively, or additionally, the one or more agents which are administered to epithelial cells may comprise or consist of recombinant plectin or an actin binding fragment thereof, and/or the one or more agents which are administered to endothelial cells may comprise or consist of an anti-plectin antibody. The recombinant plectin may comprise or consist of the actin binding domain (ABD) of plectin. The anti-plectin antibody may bind to the actin binding domain (ABD) of plectin.

The skilled person will appreciate that preferred features of any one embodiment and/or aspect of the invention may be applied to all other embodiments and/or aspects of the invention.

### BRIEF DESCRIPTION OF THE FIGURES

FIG. 1—shows that GalNAc binds to multiple proteins in endothelial cells using 1D western blot. A. Total endothelial cell protein was extracted and run on SDS-PAGE then western blot performed. Membrane on the left was probed with GalNAc-BSA-biotin and revealed several bands. As a negative control the membrane on the right was labelled with BSA-biotin and no bands were observed.

FIG. 2—shows bands excised for analysis by mass spectrometry. 1D SDS-PAGE and blot indicating where gel fragments were excised for mass spec analysis, 5 fragments were analysed.

FIG. 3—shows that GalNAc binds to multiple proteins in endothelial cells using 2D PAGE. 2D SDS-PAGE and blot indicating where gel fragments were excised for mass spec, 13 fragments in total.

FIG. 4—shows that EVs can increase cellular adhesion. EVs were isolated from breast cancer cells and incubated with either breast cancer cells, endothelial cells, or both, prior to the static adhesion assay. Breast cancer cell adhesion to endothelial cells is significantly increased in the presence of EVs. The largest increase is seen when both breast cancer cells and endothelial cells are pre-treated with EVs. Data from three replicates, error bars are standard error of the mean (SEM) and statistical test performed is one-way ANOVA with Tukey test. Star indicates significance compared to untreated control.

FIG. 5—shows that knockdown of plectin reduces adhesion of breast cancer cells to endothelial cells. MCF7 and/or HUVEC cells were treated with negative scrambles siRNA or siRNA targeting plectin. Cell adhesion is significantly decreased when plectin is knocked down in either cell line or both cell lines. Data from three replicates, error bars are standard error of the mean and statistical test performed is one-way ANOVA with Tukey test. Star indicates statistical significance compared to untreated control.

FIG. 6—shows that treatment of breast cancer cells with recombinant plectin reduces cell adhesion to endothelial cells. MCF7 cells were untreated or treated with 0.1 ug/ml recombinant plectin before performing a static adhesion assay. Treatment of MCF7 cells with recombinant plectin significantly decreases cell adhesion to endothelial cells. Data from three replicates, error bars are standard error of the mean and statistical test performed is one-way ANOVA with Tukey test.

FIG. 7—demonstrates that in cancer cells with plectin knocked down, cell adhesion can be partially recovered with EVs from ‘wild-type’ cancer cells. Plectin was knocked down using siRNA in MCF7 cells (second column) displays reduced adhesion to endothelial cells. Plectin-knockdown MCF7 cells treated with EVs from wild-type MCF7 cells show a partial recovery in adhesion to endothelial cells. Data from three replicates, error bars are standard error of the mean and statistical test performed is one-way ANOVA with Tukey test.

FIG. 8—shows that MCF7 cells have more HPA-binding proteins than BT474 cells. Lysate from the two breast cancer cell lines was run on SDS-PAGE before western blotting using biotinylated-HPA as a probe and anti GAPDH antibody as used as a loading control. MCF7 lysate contained a greater number of HPA-binding bands than lysate from BT474 cells, which only had one HPA-binding band at 72 kDa.

FIG. 9—shows a positive correlation between HPA binding capacity in cells and HPA binding capacity of EVs produced by those cells. Nanoview analysis of MCF7 and BT474 cells and EVs produced by these cells. HPA negative BC cell line (BT474, left) and HPA-positive BC cell line (MCF7, right) EVs were labelled with fluorescently conjugated antibodies (first column=CD81, second column=CD9, third column=HPA). BT474 EVs were found to be HPA negative while MCF7 EVs were HPA positive.

FIG. 10—demonstrates that blocking cell surface actin reduced breast cancer cell adhesion to endothelial cells. MCF7 cells were untreated or treated with an antibody against actin or GAPDH. Compared to non-treated MCF7 BC cells, cells incubated with anti-actin antibodies adhere significantly less to endothelial cells. Data from three replicates, error bars are SEM.

FIG. 11—shows that cell surface actin levels correlate with HPA-binding capacity/metastatic potential. Flow cytometry was used to detect surface actin. (A): shows expression of actin on the surface of highly metastatic and HPA-positive MCF7 BC cells is greater than on against non-metastatic and weakly HPA-positive BT474 cells, as well as moderately invasive and moderately HPA-positive ZR751 BC cells. Expression of actin on the surface of highly metastatic and HPA-positive MCF7 BC cells is comparable to invasive and highly HPA-positive T47D BC cells. (B) shows expression of surface actin on MCF7 and BT474 cells is much higher than on HPA-negative, normal breast cells HME.

FIG. 12—shows that actin antibody treatment compared to control antibody reduces positive single iRFP-MCF7 metastatic cells found in the bone marrow (BM) detected by flow cytometry.

FIG. 13—shows that plectin antibodies reduce metastatic cell adhesion to endothelial cells. FIG. 13 shows the results of a static adhesion assay using three plectin antibodies (PN753—Cosmo Bio, PN643—Kerafast, and Q0754—Creative Diagnostics), respectively, to block cancer cell adhesion to endothelial cells. Plectin antibodies reduce MCF7 (aggressively metastatic) (left column) cells adhering to endothelial cells compared to BT474 (right column). Based on 12 replicates. Error bars are standard error of the mean.

FIG. 14—shows that actin and/or plectin antibodies reduce metastatic cell adhesion to endothelial cells. FIG. 14 shows the result of a static adhesion assay using two actin antibodies and one plectin antibody to block cancer cell adhesion to endothelial cells. All antibodies reduce MCF7 (metastatic) cells adhering to endothelial cells. Actin 1; mAbcam8226, Actin 2; 15G5A11-E2, plectin; PN753. IgG Isotype Control; ab172730. The data is based on 12 replicates. Error bars are standard error of the mean.

FIG. 15—shows that a peptide can be used to reduce metastatic cell adhesion to endothelial cells. FIG. 15 shows the results of a static adhesion assay using a peptide and corresponding peptide conjugate to block cancer cell adhesion to endothelial cells. SEQ7 specifically reduces MCF7 (metastatic) cells binding to endothelial cells in a concentration dependant manner, compared to BT474 (non-metastatic) cells. The data is based on 12 replicates. Error bars are standard error of the mean.

FIG. 16—shows that actin antibodies reduce metastatic cell adhesion to endothelial cells. FIG. 16 shows the results of a static adhesion assay using four actin antibodies to demonstrate that metastatic MCF7 breast cancer cell binding to endothelial cells is significantly reduced when any of the actin antibodies tested were incubated with the cancer cells or both the cancer cells and the endothelial cells. ACTB1; 2-2.1.14.17 (Sigma), ACTB2; 2A3 (BioRad), ACTB3; 15G5A11/E2 (ThermoFisher), ACTB4; mbcam8226 (Abeam), IgG Isotype Control; ab172730 (Abeam). The data is based on 12 replicates. Error bars are standard error of the mean.

Materials and Methods

Cell Culture

Cells were maintained at 37° C. with 5% CO₂ atmosphere under the following conditions.

| Cell line | Basal medium | Supplements |
|---|---|---|
| MCF7 | DMEM:F-12 | Heat inactivated foetal calf serum (FCS) |
| | (Lonza) | 10% v/v (Gibco ™) |
| | | L-Glutamine 2 mM (Gibco ™) |
| T47D | DMEM:F-12 | Heat inactivated foetal calf serum (FCS) |
| | (Lonza) | 10% v/v (Gibco ™) |
| | | L-Glutamine 2 mM (Gibco ™) |
| ZR751 | RPMI-1640 | Heat inactivated FCS 10% v/v (Gibco ™) |
| | (Gibco ™) | L-Glutamine 2 mM (Gibco ™) |
| | | Sodium pyruvate 1 mM (Sigma-Aldrich ®) |
| BT474 | RPMI-1640 | Heat inactivated FCS 10% v/v (Gibco ™) |
| | (Gibco ™) | L-Glutamine 2 mM (Gibco ™) |
| | | Insulin 0.1% v/v (Sigma-Aldrich ®) |
| HME | DMEM:F-12 | Heat inactivated FCS 10% v/v (Gibco ™) |
| | (Lonza) | L-Glutamine 2 mM (Gibco ™) |
| | | Insulin 0.1% v/v (Sigma-Aldrich ®) |
| | | EGF 0.02% v/v (Peprotech) |
| | | Hydrocortisone 0.4% v/v (Sigma-Aldrich) |
| HUVEC | EBM-2 | Hydrocortisone 0.4% v/v (Lonza) |
| | (Lonza) | Human fibroblast derived growth factor-B |
| | | (hFGF-B) 0.1% v/v (Lonza) |
| | | Vascular endothelial growth factor |
| | | (VEGF) 0.1% v/v (Lonza) |
| | | R3-insulin derived growth factor-1 |
| | | (R3-IGF-1) 0.1% v/v (Lonza) |
| | | Ascorbic acid 0.1% v/v (Lonza) |
| | | Human epidermal growth factor (hEGF) |
| | | 0.1% v/v (Lonza) |
| | | Foetal bovine serum 2% v/v (Lonza) |
| | | Gentamycin and amphotericin (GA-1000) |
| | | 0.1% v/v (Lonza) |

Protein Extraction and Quantification from Endothelial Cells

HUVEC cells were grown in T175 flasks to confluence and then treated with 10 ug/ml TNFα in complete growth medium for 2 hrs at 37 C in a humidified atmosphere. Cells were washed with ice-cold PBS. Cells were pelleted by brief centrifugation, supernatant was discarded and the pellet resuspended in 1×RIPA buffer with added 10 ul/ml of protease inhibitor cocktail and placed on an end-over-end mixer at 4 C for 30 mins. After this time the tube was centrifuged at 13,000 RPM at 4° C. for 20 mins. The supernatant was transferred to a fresh 1.5 ml tube and the pellet discarded. Extracted proteins were quantified using a BCA assay.

SDS-PAGE and Western Blot

10 ug of protein sample was used for SDS-PAGE with 3 ul of Laemmli sample buffer and 1 ul of 1M DTT. This was mixed and heated to 100 C for 10 min then placed on ice. The sample was run on a mini-PROTEAN TGX stain-free precast gel (12%) in 1×TGS buffer, with Precision Plus Protein Dual Colour Standard. The gel was run at 100 volts for 2 hrs. The proteins were transferred by using a Trans-Blot Turbo Midi PVDF transfer pack. The gel was placed on the membrane and sandwiched between the layers, then placed in the Transblot Turbo machine on high MW setting. The membrane was washed in TBST (0.05% Tween) for 5 min on a shaking platform, three times. The membrane was then blocked by incubating with 5% BSA/TBST for 2 hrs on a shaking platform RT. Blocked membrane was incubated in 10 ml of 1 ug/ml GalNAc-BSA-Biotin in 5% BSA/TBST or 10 ml of 1 ug/ml BSA-Biotin in 5% BSA/TBST overnight at 4 C. Membranes were washed with 5% BSA/TBST for 5 min three times, before incubating the membrane in 10 ml of 5 ug/ml streptavidin-HRP in 5% BSA-TBST at RT for 1 hr. The membrane was washed with 5% BSA-TBST for 5 mins twice then once for 15 mins. HRP substrate was prepared (Clarity+Clarity Max Western ECL Substrate, BioRad, 170-5060) in a 1:1 ratio and mixed. This was added to the membrane and imaged on a Chemidoc® transilluminator.

2D PAGE

Activated total HUVEC protein at a concentration of 1 ug/ul in 1×RIPA buffer with added protease inhibitors was cleaned-up for use in 2D PAGE by using 2D Clean-up kit from GE Healthcare, according to manufacturer's instructions (80-6484-51). The sample was prepared for 2D PAGE by using ReadyPrep 2-D starter kit from BioRad according to manufacturer's instructions (163-2105). This allowed the sample to be absorbed by the 11 cm IPG strips and then subsequently separated by isoelectric-focusing in Ettan IPGphor 3 for 16 hrs. The IPG strips were washed and rehydrated then placed in a Criterion™ XT Bis-Tris Precast Gels, 12% Gels were loaded into tanks and standard added and gels sealed with agarose. Agarose was allowed to set before filling tank with 1×MOPS buffer. 250 ul of NuPage Antioxidant (Thermo, NP0005) was added per gel. Gels were run at 200 volts for 70 min. Gel to be blotted was removed from its cassette and washed twice in dH₂O. The remaining procedure was carried out at room temperature. The gel was placed in Invitrogen™ Novex™ SimplyBlue™ SafeStain (Thermo, LC6065) and placed on a shaking platform for 1 hr. The gel was then de-stained in dH₂O overnight on a shaking platform. The gel was blotted using the iBlot™ 2 Dry Blotting System. Membrane was washed briefly in dH2O and then blocked with 5% Marvel/TBST for 1 hr on a rocking platform. The membrane was incubated with 1 ug/ml GalNAc-BSA-Biotin in 5% BSA/TBST for 2 hr on a rocking platform and then washed 3 times for 10 min each with TBST on a rocking platform. The membrane was incubated with 5 ug/ml Streptavidin-peroxidase in 5% BSA/TBST for 1 hr on a rocking platform and then washed 3 times for 10 min each with TBST on a rocking platform. ECL HRP substrate was prepared and added to the membrane and then imaged using Chemidoc® transilluminator.

Preparation of Gels for Mass Spectrometry

1D gels were stained with Coomassie blue and 2D gels were stained with silver stain and then bands and dots of interest were excised from the 1D and 2D gels using blots as a reference. Gels were then sent for mass spectrometry at Porton Biopharma Ltd.

EV Extraction from Breast Cancer Cells

MCF7 breast cancer cells were grown in T175 flasks to 70% confluency and fed with 25 ml/flask of EV cleared media (DMEM media containing 10% FBS, wherein the FBS has been previously spun for 16 hrs at 120,000 g to clear existing EVs), and conditioned for 48 hrs. Media was removed and spun at 300×g for 5 min then at 16,000×g for 20 mins at 4° C. Supernatant was filtered through a 0.22 um filter which had been blocked with 0.1% BSA. The supernatant was concentrated using a Vivaspin 20, 100 kDa concentrator to 500 ul. SEC columns (BioRad, 7321010) were prepared by adding 14 ml of sepharose and 10 ml of PBS this was allowed to settle for 2 hours and then a column support was added and PBS allowed to flow through. The column was then washed three times with 10 ml of PBS. 500 ul of samples was added to the column support and allowed to absorb then 10 ml of PBS added. 2.5 ml of flow-through was collected and discarded then 2 ml of flow-though was collected (containing EVs). EVs were quantified using Particlematrix™ particle analyser.

Endothelial Static Adhesion Assay with or without EVs

Coverslips with activated endothelial cells were prepared as above. MCF7 breast cancer cells were grown in T75 flasks until 70% confluency and treated with 10 mg/ml 8-hydroxypyrenetrisulphonic acid (HTPS) in complete medium for 2 hrs at 37° C., 5% CO₂. After this time, HTPS was removed and cells were washed with 10 ml of PBS 5 times until the wash ran clear. Cells were scraped and counted. 20,000 cells/ml was prepared. MCF7 cells were treated with either 500 ul of EVs or 500 ul of PBS and mixed for 10 mins at RT. HUVEC cells were treated with either 500 ul of EVs or 500 ul of PBS at 37° C., 5% CO₂ for 30 mins. EVs or PBS was removed from cells and 20,000 cells/ml of MCF7 cells were added per well and incubated for 10 mins, cells were removed and wells gently washed with warmed PBS to remove any unbound cells. Wells were then fixed with 4% PFA for 10 mins at RT. Fixative was removed and the wells washed 3 times with PBS and then coverslips were mounted using Fluoromount™. Total adhered cells per coverslip were counted.

Coverslips with activated endothelial cells were prepared as above. MCF7 breast cancer cells were grown in T75 flasks until 70% confluency and treated with 10 mg/ml HTPS in complete medium for 2 hrs at 37° C., 5% CO₂. HTPS was removed and cells were washed with 10 ml of PBS 5 times until the wash ran clear. Cells were scraped and counted. 20,000 cells/ml was prepared. MCF7 cells were treated with either 0.1 ug/ml recombinant plectin, (2b Scientific SKU RPC754Hu01 (residues Asp 175-Pro 400 (SEQ ID NO: 1) of human plectin Uniprot ID Q15149, corresponding to the actin-binding domain), in PBS or PBS for 10 mins at RT. Recombinant plectin or PBS were removed from cells and 20,000 cells/ml of MCF7 cells were added per well and incubated for 10 mins, cells were removed and wells gently washed with warmed PBS to remove any unbound cells. Wells were then fixed with 4% PFA for 10 mins at RT. Fixative was removed and the wells washed 3 times with PBS and then coverslips were mounted using fluoromount. Total adhered cells per coverslip were counted.

Endothelial Static Adhesion Assay with or without Plectin Silencing and EV Rescue

MCF7 and HUVEC cells were grown in 24 well cell culture plates to 70% confluency the cells were treated with Dharmafect™ transfection reagent and 5 uM of plectin siRNAs or scrambled negative control for 24 hrs. Some HUVEC and MCF7 cells were non-treated. HUVEC cells were activated by treating with 10 ug/ml TNFa in complete medium for 2 hrs at 37° C., 5% CO₂. MCF7 breast cancer cells were grown in T75 flasks until 70% confluency and treated with 10 mg/ml HTPS in complete medium for 2 hrs at 37° C., 5% CO₂. HTPS was removed and cells were washed with 10 ml of PBS 5 times until the wash ran clear. Cells were scraped and counted. 20,000 cells/ml was prepared. MCF7 cells were treated with either 500 ul of EVs isolated using the extraction method outlined above, or 500 ul of PBS for 10 mins at RT. EVs or PBS was removed and 1 ml of MCF7 cells were added to HUVEC cells and incubated at 37° C., 5% CO₂ for 10 mins, before cells were removed and wells gently washed with warmed PBS to remove any unbound cells. Wells were then fixed with 4% PFA for 10 mins at RT. Fixative was removed and the wells washed 3 times with PBS and then coverslips were mounted using fluoromount. Total adhered cells per coverslip were counted.

Endothelial Static Adhesion Assay with or without Antibodies and Peptides

This was as above but using 1/1000 of stock antibodies and peptides stated below.

Plectin 1 ab=https://www.2bscientific.com/Products/Cosmo-Bio-Ltd/CAC-NU-01-PLN/Anti-plectin.

Plectin 2 ab=https://www.kerafast.com/item/750/anti-plectin-n-terminal-actin-binding-domain-pn643-antibody.

Plectin 3 ab=https://www.creative-diagnostics.com/search.aspx?keys=cabt-b160&cid=0.

ACT1=https://www.abcam.com/beta-actin-antibody-mabcam-8226-loading-control-ab 8226.html.

ACT2=https://www.thermofisher.com/antibody/product/beta-Actin-Antibody-clone-15G5A11-E2-Monoclonal/MA1-140.

SEQ7 conjugate = AVIYGGVQGGC-HAS.
SEQ7 peptide = AVIYGGVQ.
SEQ8 conjugate = WPYKKYGGC-HAS.

ACTB1 anti-gamma https://www.sigmaaldrich.com/catalog/product/sigma/a8481?lang=en&region=GB&c m_sp=Insite-_-caSrpResults_srpRecs_srpModel_a8481-_-srpRecs3-1.

ACTB2 anti-gamma=https://www.bio-rad-antibodies.com/monoclonal/human-actin-gamma-antibody-2a3-vma00049.html?f=Antibody-only&_ga=2.15891637.2017118208.1608138841-1927149081.1608138841.

ACTB3 anti beta=https://www.thermofisher.com/antibody/product/beta-Actin-Antibody-clone-15G5A11-E2-Monoclonal/MA1-140.

ACTB4 anti beta=https://www.abcam.com/beta-actin-antibody-mabcam-8226-loading-control-ab8226.html

HPA Probing after Western Blot of MCF7 and BT474 Breast Cancer Cells

MCF7 and BT474 cell lines were grown to 70% confluence in T75 flask and had their proteins extracted, quantified, run on SDS-PAGE and then blotted as described. Membrane was probed using biotinylated-HPA or anti-GAPDH antibody.

Nanoview

Following the Nanoview manual. EVs extracted from MCF7 and BT474 breast cancer cells using SEC (as described) and labelled with fluorescently conjugated antibodies (CD81 and CD9— clones not publicly stated in the kit) as well as HPA lectin (Sigma).

Preparation of Activated Endothelial Cells on Coverslips

Sterilised 13 mm D glass coverslips were placed in wells of 24 well plate and 100,000 HUVEC cells were seeded. Cells were incubated at 37° C. with 5% CO₂ atmosphere until cells became a monolayer. HUVEC cells were activated by treating with 10 ug/ml TNFa in complete medium for 2 hrs at 37° C., 5% CO₂.

Endothelial Adhesion Assay with or without Actin Antibody Inhibition

Coverslips with activated endothelial cells were prepared as above. MCF7 breast cancer cells were grown in T75 flasks until 70% confluency and treated with 10 mg/ml HTPS in complete medium for 2 hrs at 37° C., 5% CO₂. After this time HTPS was removed and cells were washed with 10 ml of PBS 5 times until the wash ran clear. Cells were scraped and counted. 20,000 cells/ml was prepared. MCF7 cells were treated with either 1:1,000 actin IgG (Abcam ab8227), 1:1,000 GAPDH IgG or PBS for 30 mins at RT. Antibodies or PBS were removed from cells and 20,000 cells/ml of MCF7 cells were added per well and incubated for 10 mins, cells were removed and wells gently washed with warmed PBS to remove any unbound cells. Wells were then fixed with 4% PFA for 10 mins RT. Fixative was removed and the wells washed 3 times with PBS and then coverslips were mounted using fluoromount. Total adhered cells per coverslip were counted.

Flow Cytometry

Cells were harvested and resuspended in cold PBS. Cells were incubated them with anti-actin Ab for 1 h at 4° C. (Proteintech cat: 60008-1-Ig). Cells were then washed and resuspended in PBS before flow cytometry using a 13-color, 4-laser CytoFLEX S N-V-B-R Flow Cytometer, equipped with 375 nm, 405 nm, 488 nm and 638 nm lasers and operated using CytExpert Software.

Animal Study

This was run by Charles River Discovery Research Services in Germany. The experiment was set up to test the efficacy of an antibody test article to inhibit tumour cell colonization model in a mouse model. The model comprises of the breast cancer tumor cell line iRFP-MCF7, injected intravenously (iv) into NSG mice. 10 NSG mice received intravenous injections of 5×10\hat{ }6 iRFP-MCF7 cells (suspension in 300 ul PBS); the day of injection is designated as day 0. Mice were randomized into two therapy groups (5 mice per group); one group for the control antibody and one group for the actin antibody (clone 2A3). Mice were provided with estradiol in drinking water (10 μg/ml), given the MCF7 cells express the estrogen receptor and maintain the ability to process estradiol via cytoplasmic estrogen receptors (www.atcc.org). Estrogen is considered essential for in vivo growth of MCF7 cell (Gierthy J F, et al., Cancer Res. (1993): 53(13):3149-53). The experiment starts with i.p. therapies followed by i.v. injection of tumor cells 30 min later; this was defined as the starting day (d=0) of the experiment. The mice were given 30 mg/kg/d of control antibody or actin antibody dosed at 5 ml/kg by ip in a vehicle of physiological saline and treated once a week. The animals were euthanized after 20 days. Bone marrow (BM) was analysed for iRFP-MCF7 cells by flow cytometry; separating cell populations of iRFP signal, forward-sideward scatter and mCD45. These were gated for singlets.

### EXAMPLES

### Example 1—Identification of Membrane-Bound Actin as a Biomarker for Metastatic Potential

Binding of Helix pomatia agglutinin (HPA), a lectin from the Roman snail, to breast cancer (BC) and other epithelial cancers is associated with metastatic competence and poor patient prognosis (Brooks S., 2000, Histology and Histopathology, 15(1): 143-158). What HPA binds to specifically is not known, however, it has been shown that HPA binds to a range of glycoproteins with terminal α-GalNAc (the monosaccharide with which HPA has then highest affinity). One of these glycoproteins is the cancer associated Tn antigen (Ser/Thr-GalNAc) which is the initial structure made in 0-linked glycosylation which is normally always further elaborated, but is a common occurrence in cancer (Brooks S. A. & Leathem A. J. C., 1995, British J. Cancer, 71: 1033-1038).

Highly HPA-positive BC cells adhere significantly more to endothelial cells in a static adhesion assay than weakly HPA-positive or HPA-negative BC cells. This assay mimics metastatic cancer cell arrest in narrow capillaries. For cancer cells to successfully metastasise they must bind to and extravasate out of the blood vessel. It has been shown that binding is mediated through this glycosylation mark as incubating cells with HPA or BSA-GalNAc inhibits cells binding.

Identification of the Receptor/s on the Endothelial Cells which Mediate HPA-Positive Cell Adhesion.

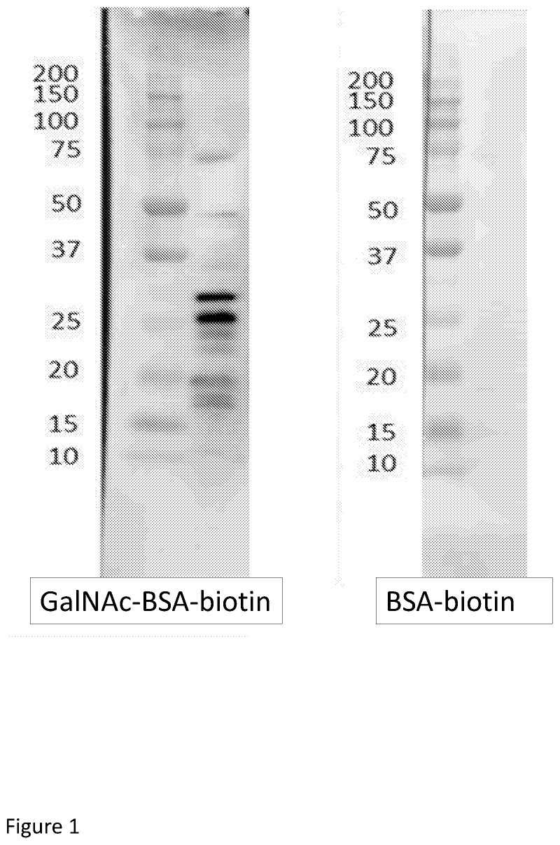

In order to identify the putative receptor/s on endothelial cells which are recognising the HPA-binding glycans on the BC cells, total protein was extracted from the endothelial cells that had been activated using TNFα (an inflammatory cytokine known to upregulate endothelial receptors). Protein was run on SDS-PAGE and then blotted. GalNAc-BSA-biotin (GalNAc is the sugar with which HPA has the greatest nominal specificity for) was used as a probe to identify endothelial proteins of interest. As a negative control BSA-biotin was also used to probe the membrane. As illustrated in FIG. 1, when the membrane was probed with GalNAc-BSA-biotin several bands were observed, whilst no bands were observed when the membrane was probed with BSA-biotin.

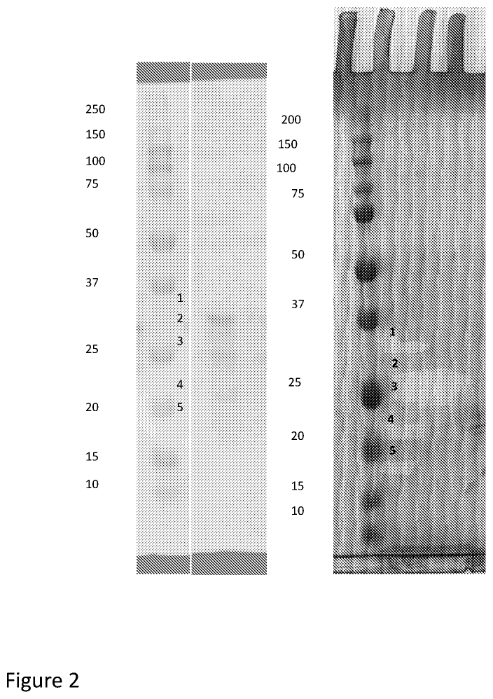

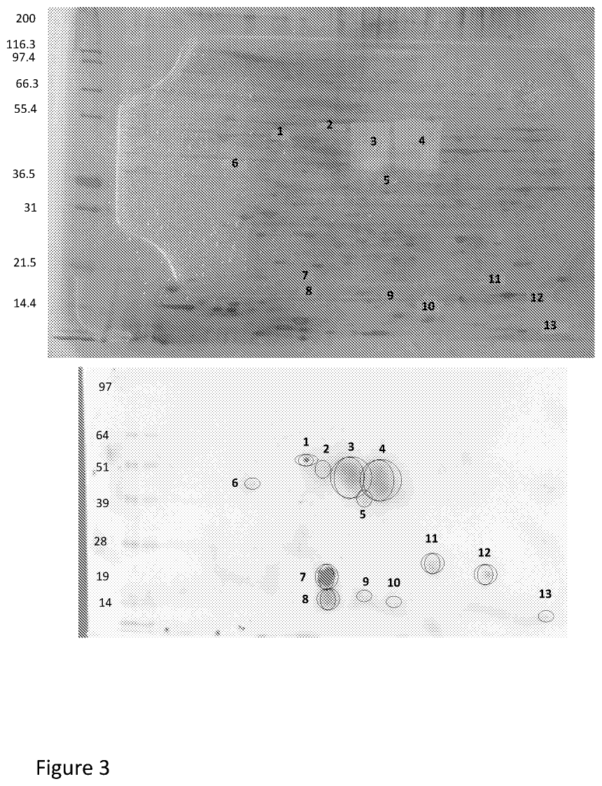

Several bands were consistently identified using GalNAc-BSA-biotin as a probe, from these, five gel fragments were selected and excised for mass spec (FIG. 2). Bands observed in blot from 1D gel could represent a large number of proteins as separation is only on size. To better resolve the bands, 2D-PAGE was used to separate proteins on both size and pH. 2D gels were blotted and probed with GalNAc-BSA-Biotin. Thirteen spots were identified as of interest and were excised from the 2D PAGE gel (FIG. 3).

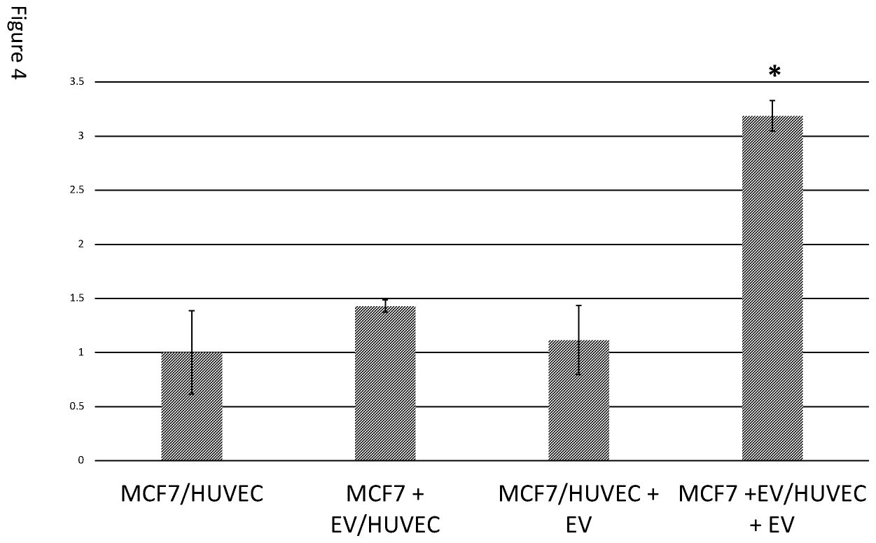

Mass spec data revealed several proteins in the gel fragments from both 1D and 2D-PAGE, some of which overlapped in both. Amongst those proteins identified was plectin, which is normally an intracellular scaffold protein. However, Shin et al. (2013) demonstrated that plectin can localise to the membrane of cells through exosome secretion. To investigate this, EVs were isolated from BC cells and incubated with either BC cells, endothelial cells or both prior to the static adhesion assay. It was found that incubating both the BC cells and endothelial cells with EVs significantly increased binding of BC cells to endothelial cells when compared to non-treated, BC cell only or endothelial cell only treated (FIG. 4). This finding suggested that EVs may have a positive role in aiding BC cell adhesion to endothelial cells.

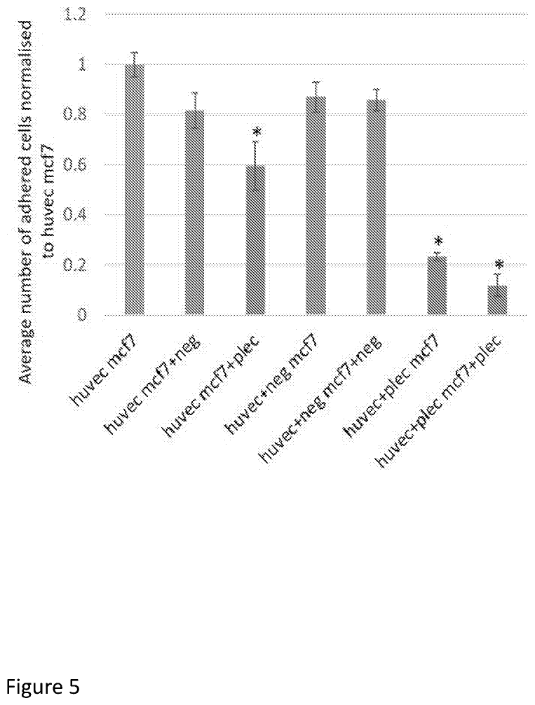

To investigate this further, plectin was knocked down in either the BC cells, endothelial cells or both, using siRNA. In all instances, binding of BC cells to endothelial cells was significantly reduced when compared to both non-treated and scrambled siRNA treated cells (termed neg on graph) negative control cells, therefore suggesting that plectin is needed for BC cells to bind to endothelial cells (FIG. 5).

Confirmation that the Receptor on the Endothelial Cells is Specific for the Glycosylation Marks on the BC Cells and Mediates Cell Binding

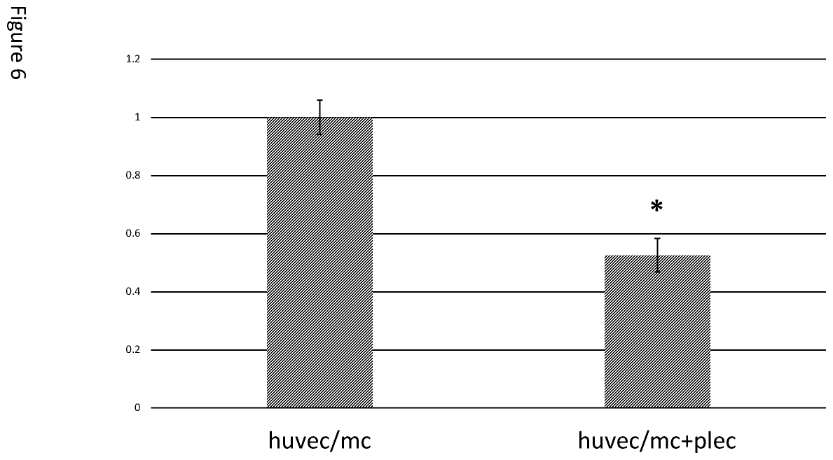

To confirm that the receptor on the endothelial cells is specific for the glycosylation mark on the BC cells, a recombinant plectin fragment (containing the actin-binding domain of plectin, which can bind all actin isoforms (Fontao et al., 2001, J. Cell. Sci., 114: 2065-2076)) was used to block BC cells, by incubating for 10 mins prior to addition of the endothelial cells. Cell adhesion to the endothelial cells was significantly reduced when compared to non-treated control cells (FIG. 6). It was hypothesized that the reduced binding observed when incubating the BC cells with the recombinant plectin is a result of the plectin binding to the HPA-positive glycosylation mark, thereby “capping” them and preventing binding to the endothelial cells.

Investigation of how EVs from BC Cells Increase Adhesion to Endothelial Cells

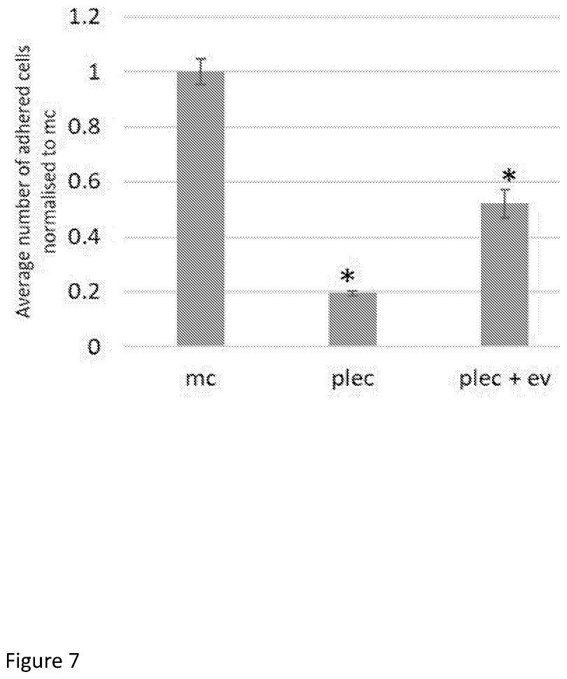

The findings that knockdown of plectin in the BC cells, combined with the observation that EVs from BC cells can increase the adhesion of BC cells to endothelial cells, led to the hypothesis that EVs can carry plectin from BC cells to the surface of endothelial cells, which then increase the effective “landing platform” for BC cells to bind to. To begin testing this hypothesis, a “rescue” experiment was performed. Binding of BC cells to endothelial cells was reduced when plectin was knocked down in BC cells (FIG. 7). Interestingly, this reduced binding was partially rescued when endothelial cells were treated with EVs from control BC cells. This is consistent with the hypothesis that the EVs from the normal cells (which have plectin) are providing the “landing platform” to allow binding to occur.

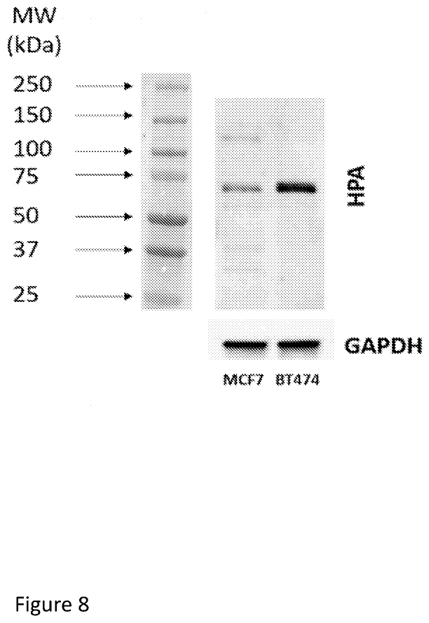

In a model system, two BC cell lines were used which have been reported to be highly HPA positive (MCF7) and weakly HPA positive (BT474) to verify the above hypothesis. Protein was extracted from the two cell lines and run on SDS-PAGE then blotted and probed with HPA. The HPA-positive BC cell line (MCF7) possessed a greater range of HPA positive glycoproteins than the weakly HPA-positive BC cell line (BT474), which has only one band at 75 kDa, thereby confirming the use of this model system (FIG. 8).

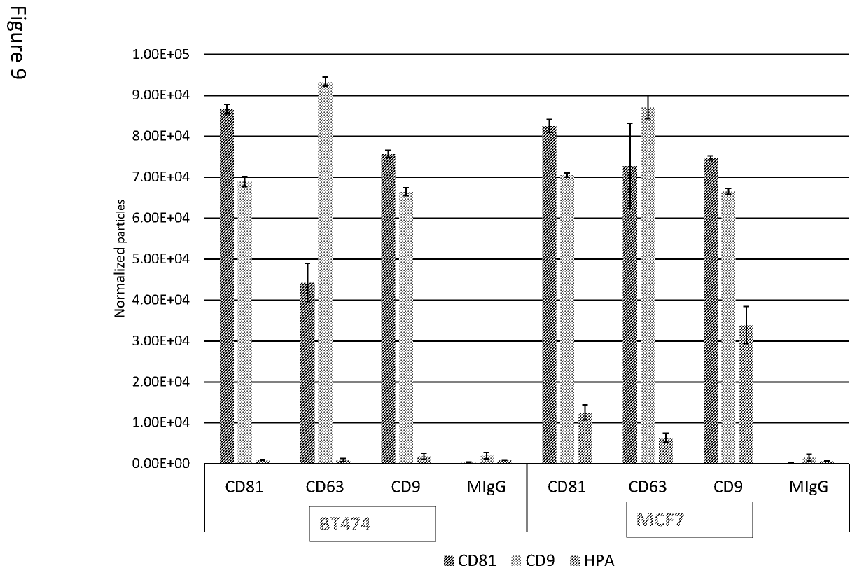

Using NanoView, it was shown that HPA-binding-positive cells produce HPA-binding-positive EVs and likewise HPA-binding-negative cells produce HPA-binding-negative EVs. To do this, EVs were isolated from two BC cell lines, one HPA-binding-negative and the other HPA-binding-positive, by labelling the two EV populations with fluorescently conjugated antibodies specific for two commonly used EV markers and HPA it was possible to quantify the amount the EVs had ‘HPA-binding-positivity’. EVs from HPA-binding-negative BC cells (BT474 left, FIG. 9) were found to be HPA-binding-negative whist EVs from HPA-binding-positive BC cells (MCF7 right, FIG. 9) were HPA-binding-positive. In particular, HPA was predominately colocalized with CD9 on vesicles—a maximum of 45% of CD9 positive vesicles are HPA positive compared to 13% of CD81 positive and 9% of CD63 positive vesicles. Due to overlap with the APC emission spectrum, fluorescently conjugated anti-CD63 was not included in this experiment.

Identification of the Glycoprotein on BC Cells which Mediates Binding to the Receptor on the Endothelial Cells

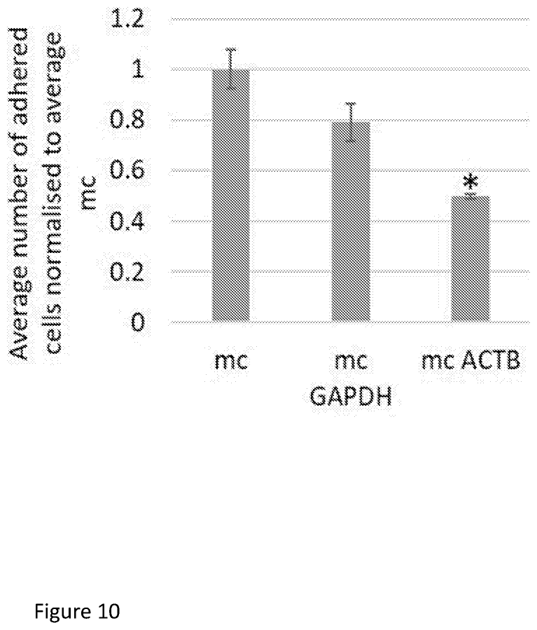

Cytoskeletal plectin has a number of binding partners which interact with its actin-binding domain (ABD), including actin. To investigate whether glycosylated actin is a potential binding partner of cell surface plectin, a blocking experiment was performed, where actin was masked using an anti actin IgG (Abcam ab8227) for 30 mins prior to incubation with endothelial cells). Compared to untreated control cells, when actin is blocked, cell adhesion is significantly reduced, therefore suggesting that actin is a binding-partner for plectin and that this interaction at contributes to the binding of BC cells to endothelial cells (FIG. 10).

Levels of Surface Actin on Different Cell Lines

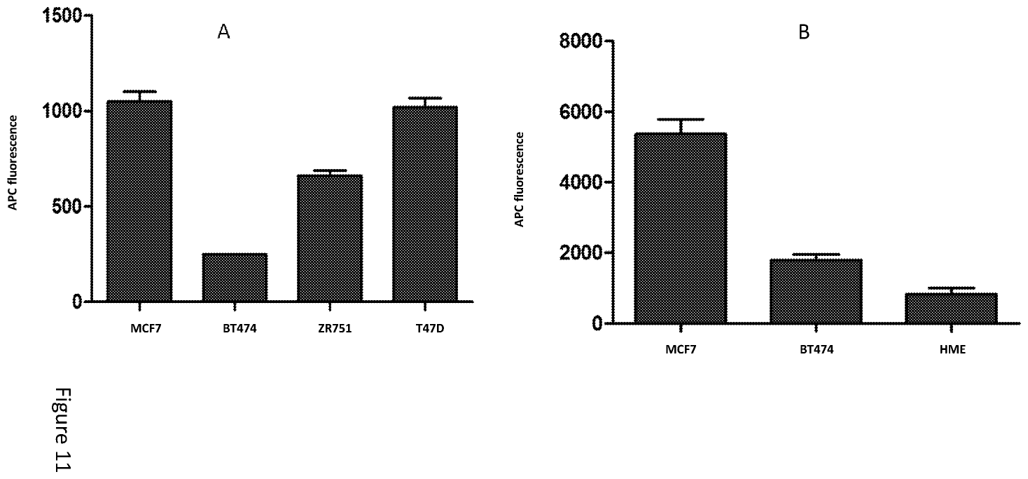

A range of cells with known invasiveness and HPA-binding profiles were used to determine the level of surface actin on different cell types by flow cytometry. These ranged from normal/HPA-negative, to non-metastatic/weakly HPA-positive to highly metastatic/highly HPA-positive (FIG. 11). HME cells are normal breast cells and are HPA-negative; BT474 is derived from a primary invasive ductal cancer, with no evidence of metastases (Lasfargues et al. 1978, J. Nat. Cancer Institute, 61(4): 967-978) and labels weakly with HPA; ZR 751 was derived from malignant ascites from an invasive primary ductal breast cancer—i.e. from cells that had already metastasised (Engel et al. 1978, Canc. Res., 38, 3352-3364) and labels moderately with HPA; T47D was derived from malignant pleural effusion from an invasive primary ductal breast cancer—i.e. from cells that had already metastasised (Keydar et al 1979, Eur J Cancer, 15(5):659-70) and labels highly with HPA; MCF7 was derived from malignant pleural effusion from an invasive primary ductal breast cancer—i.e. from cells that had already metastasised (Soule et al. 1973, J. Nat. Cancer Institute, 51(5): 1409-14-16) and labels highly with HPA. Actin labelling appears to follow a similar trend as HPA labelling, in that increased surface actin is measured in more invasive/metastatic cells.

### Example 2—Demonstration that Actin and/or Plectin Antibody Treatment Reduces Metastatic Cell Colonization/Adhesion

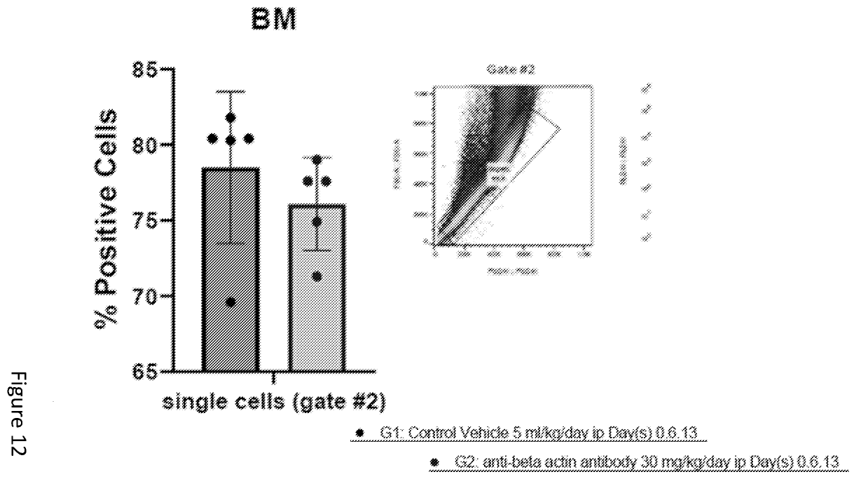

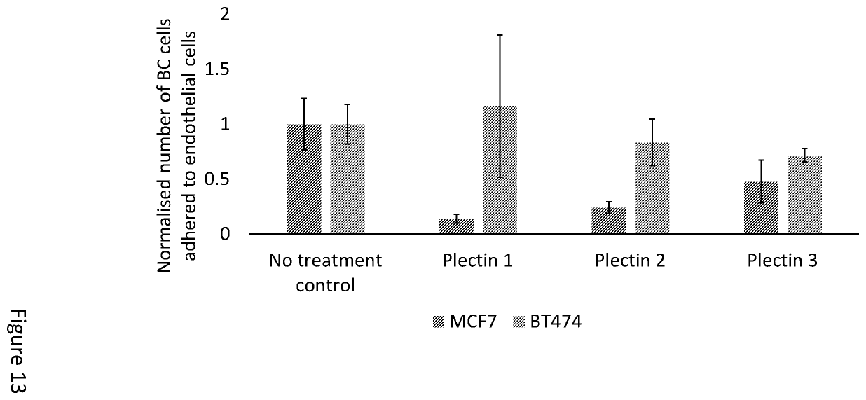

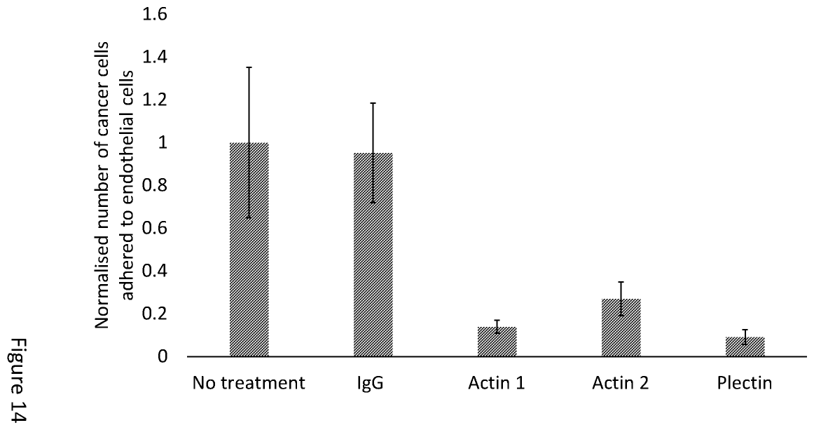

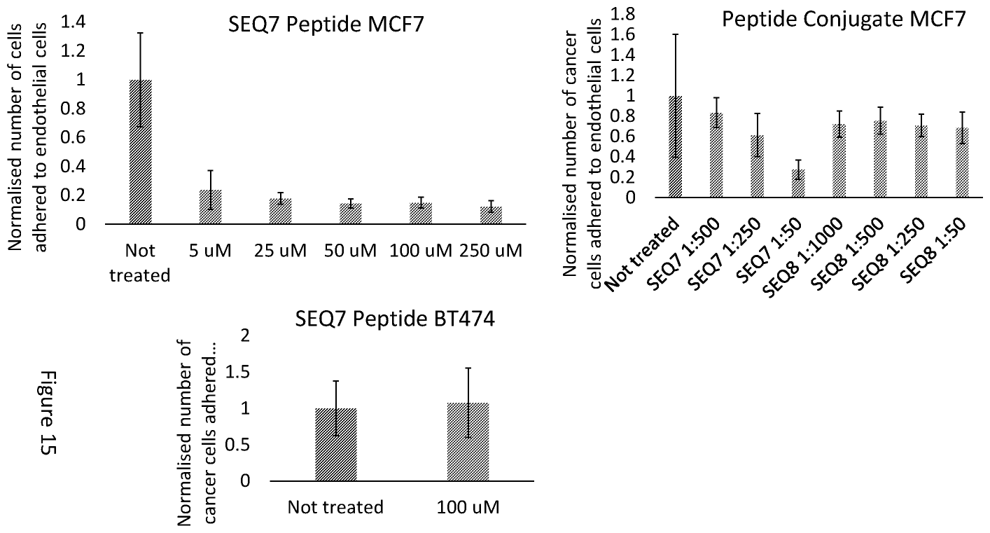

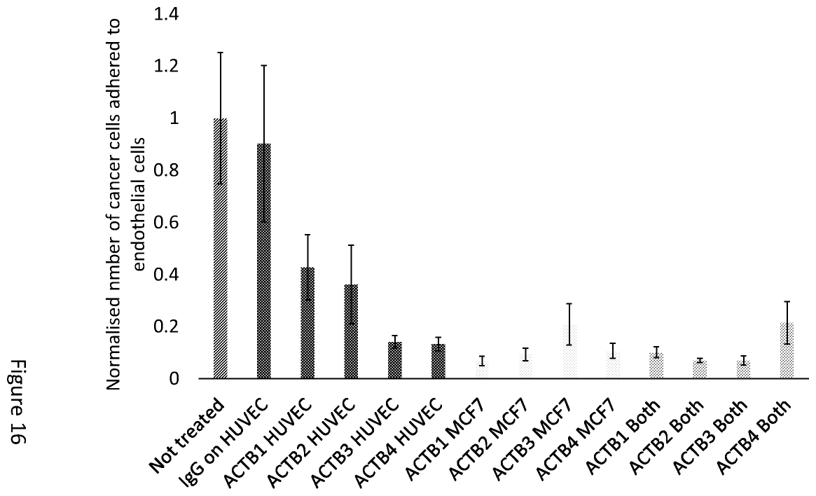

Metastatic breast cancer cell colonization model in a mouse model is shown to be reduced upon treatment with an anti-actin antibody (FIG. 12). Similarly, metastatic breast cancer cell adhesion to endothelial cells is reduced upon treatment with three different plectin antibodies (FIG. 13). A similar study demonstrates that metastatic breast cancer cell adhesion to endothelial cells is reduced upon treatment with either of two different actin antibodies or a plectin antibody (FIG. 14). Additionally, other reagents, in the case of peptides, are also shown to be able to reduce metastatic breast cancer cell adhesion to endothelial cells (FIG. 15). Further, use of different actin antibodies incubated either cancer cells, endothelial cells or both cancer and endothelial cells reduces breast cancer cell binding to endothelial cells (FIG. 16). Collectively, the data demonstrates that inhibition of the interaction between membrane-bound actin and plectin and/or blocking membrane-bound actin or plectin reduces metastatic cell adhesion to endothelial cells, which is a biological phenomenon required for metastasis to occur.

### Discussion

In conclusion, blocking or reducing the level of interaction between actin or one of its interacting partners such as plectin or an actin binding fragment thereof on the surface (membrane-bound) of epithelial cancer cells or EVs, or reduction of the level of expression of the one or more interacting partners, advantageously serves as a prophylactic or therapeutic treatment for metastatic cancer, and in particular metastatic cancers derived from primary epithelial cell cancers such as breast cancer. This is demonstrated particularly in FIGS. 6, 7 10 and 12-15.

1. An agent which prevents or reduces the interaction between actin and one or more of its interacting partners, for use in treating or preventing metastatic cancer in a subject in need thereof.

2. The agent for use according to claim 1, wherein the one or more interacting partners comprises or consists of plectin or an actin-binding fragment thereof.

3. The agent for use according to claim 1 or claim 2, wherein the agent is a polypeptide, a small molecule, an antigen binding polypeptide, or a nucleic acid molecule.

4. The agent for use according to any of claims 1-3, wherein the agent binds to actin which is present on the surface of a cell and/or EVs.

5. The agent for use according to any of claims 1-4, wherein the agent is an anti-actin antibody.

6. The agent for use according to any of claims 1-5, wherein the agent is an anti-beta-actin antibody.

7. The agent for use according to any of claims 1-3, wherein the agent comprises or consists of plectin or an actin-binding fragment thereof, or recombinant actin.

8. The agent for use according to claim 7, wherein the actin binding fragment thereof comprises or consists of the actin binding domain (ABD) of plectin (SEQ ID NO: 1 or SEQ ID NO: 2).

9. The agent for use according to any of claims 1-3, wherein the agent binds to plectin which is present on the surface of a cell.

10. The agent for use according to any of claim 1-3 or 9, wherein the agent is an anti-plectin antibody.

11. The agent for use according to claim 10, wherein anti-plectin antibody binds the actin binding domain (ABD) of plectin.

12. The agent for use according to any of claims 1-11, wherein the metastatic cancer is an epithelial cell cancer metastasis.

13. The agent for use according to claim 12, wherein the epithelial cell cancer metastasis is a breast cancer metastasis.

14. A pharmaceutical composition for use in treating or preventing metastatic cancer in a subject in need thereof, comprising an agent for use according to any of claims 1-13.

15. The pharmaceutical composition for use according to claim 14, comprising two or more different agents for use according to any of claims 1-13.

16. The pharmaceutical composition for use according to claim 15, wherein a first agent binds to actin which is present on the surface of a cell, and a second agent binds to plectin or an actin-binding fragment thereof which is present on the surface of a cell.

17. The pharmaceutical composition for use according to any of claims 14-16, further comprising one or more carriers or excipients.

18. A method of treating or preventing metastatic cancer, comprising administering to a subject in need thereof a therapeutically effective amount of one or more agents wherein the agent is as defined in any of claims 1 to 13.

19. A method of preventing or reducing epithelial cell to endothelial cell adhesion comprising administering to a subject, or to one or more cells, one or more agents as defined in any of claims 1 to 13.

20. The method according to claim 18 or claim 19, wherein the one or more agents are administered to epithelial cells and/or the one or more agents are administered to endothelial cells.

21. The method of claim 20, wherein the one or more agents which are administered to epithelial cells comprise or consist of an anti-actin antibody, and/or the one or more agents which are administered to endothelial cells comprise or consist of an anti-plectin antibody.

22. The method of claim 20, wherein the one or more agents which are administered to epithelial cells comprise or consist of recombinant plectin or an actin binding fragment thereof, and/or the one or more agents which are administered to endothelial cells comprise or consist of an anti-plectin antibody.

23. The method of claim 22, wherein the recombinant plectin comprises or consists of the actin binding domain (ABD) of plectin.

24. The method of any of claims 21-24, wherein the anti-plectin antibody binds the actin binding domain (ABD) of plectin.

25. The method of any of claim 18 wherein the metastatic cancer is an epithelial cell cancer metastasis, optionally the epithelial cell cancer metastasis is a breast cancer metastasis.
